(19) United States
(12) Patent Application Publication
(10) Pub. No.: US 2011/0190333 A1
(43) Pub. Date: Aug. 4, 2011

(54) DIHYDROTETRABENANZINE FOR THE TREATMENT OF ANXIETY

(75) Inventors: Ben Grayson, West Yorkshire (GB); Roger Anthony Oakes, Rutland (GB); Andrew John Duffield, Hertfordshire (GB)

(73) Assignee: Biovail Laboratories International (Barbados) S.R.L., Christ Church (BB)

(21) Appl. No.: 12/997,258

(22) PCT Filed: Jun. 12, 2009
(86) PCT No.: PCT/GB2009/050670

§ 371 Date: Mar. 25, 2011

(30) Foreign Application Priority Data
Jun. 13, 2008 (GB) 0810857.3

(51) Int. Cl. A61K 31/4745; A61P 25/22

(52) U.S. Cl. 514/294

(57) ABSTRACT

The invention provides a 3,11b-cis-dihydrotetrabenazine of the formula (Ib): or a pharmaceutically acceptable salt thereof for use in the prophylaxis or treatment of anxiety.

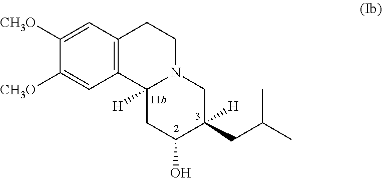

This invention relates to a dihydrotetrabenazine for use in the prophylaxis or treatment of anxiety.

### BACKGROUND OF THE INVENTION

Anxiety is a physiological state characterized by a combination of cognitive, somatic, emotional, and behavioral components. Whereas it is a common emotion along with fear, anger, sadness, and happiness, and has a very important function in relation to survival, it can become pathological or maladaptive in some people.

Anxiety is often manifested as anger, fear, apprehension, or worry, and people suffering from anxiety may find that that they easily lose their patience, have difficulty concentrating, think constantly about the worst outcome in a given situation, have difficulty sleeping, become depressed and/or develop obsessive behaviour.

The mental symptoms of anxiety are frequently accompanied by physical symptoms such as heart palpitations, pale skin, sweating, nausea, chest pain, shortness of breath, stomach aches, headache, excessive thirst, flatulence, diarrhoea, increased frequency of urination, sexual impotence, muscle pain, dizziness, pins and needles, tremors and painful or absent periods.

With the exception of panic attacks which are unpredictable and self limiting, anxiety can be generally classified according to cause or the circumstances in which the anxiety arises. One scheme that has been suggested classifies types of anxiety as follows:

1. Anxiety secondary to other psychiatric illness.
2. Primary anxiety
Anxiety neurosis which may have genetic influences.
Phobic anxiety which includes social phobias, general phobias and specific phobias.
3. Anxiety resulting from obsessional disorders.

Excessive anxiety and anxiety disorders can be treated with anxiolytic drugs, examples of which are selective serotonin reuptake inhibitors (SSRIs), benzodiazepines and beta-receptor blockers such as propranolol and oxprenolol (which although not anxiolytics per se, can be used to combat the somatic symptoms of anxiety).

Examples of SSRIs include citalopram (Celexa, Cipramil, Emocal, Sepram, Seropram), escitalopram (Lexapro, Cipralex, Esertia), fluoxetine (Prozac, Fontex, Seromex, Seronil, Sarafem, Fluctin (EUR), Fluox (NZ)), fluvoxamine (Luvox, Faverin, Dumyrox), paroxetine (Paxil, Seroxat, Aropax, Deroxat, Rexetin, Xetanor, Paroxat), sertraline (Zoloft, Lustral, Serlain), and zimelidine (Zelmid, Normud), all of which are associated with any of a variety of adverse side effects.

Examples of benzodiazepines include lorazepam (Ativan), clonazepam (Klonopin), alprazolam (Xanax), and diazepam (Valium) and these are typically prescribed for short-term relief of severe and disabling anxiety. Benzodiazepines may also be administered to cover the latent periods associated with drugs prescribed to treat an underlying anxiety disorder. Benzodiazepines are also used as a longer term treatment for severe anxiety. However, there are problems associated with the use of benzodiazepines, namely the risk of withdrawal symptoms and the risk of rebound syndrome after continuous usage of more than two weeks. In addition, there is the added potential problem of the accumulation of drug metabolites and adverse effects.

Buspirone (BuSpar) is a serotonin 1A agonist which lacks the sedative side effects and the dependence associated with benzodiazepines and causes much less cognitive impairment. However, a disadvantage of buspirone is that 1 to 3 weeks can often elapse following administration before the anxiolytic effect of the drug becomes evident.

Other anxiolytics used in the past include barbiturates and meprobamate which exert an anxiolytic effect linked to the sedation they cause. However, the risk of abuse and addiction to these drugs is high and they are now largely obsolete as medications for treating anxiety.

Thus, at the present time, there remains a need for alternative anxiolytic agents which lack all or some of the side effects associated with known anxiolytics.

Our earlier International patent applications WO/2007/017654, WO/2007/017643, WO/2007/007105 and WO/2005/077946 disclose the preparation and use of dihydrotetrabenazine isomers for various therapeutic uses including the treatment of hyperkinetic movement disorders such as Huntington's disease, hemiballismus, senile chorea, tic, tardive dyskinesia and Tourette's syndrome; the treatment of Huntington's disease, the treatment of inflammatory diseases and the treatment of psychoses such as schizophrenia.

The dihydrotetrabenazine isomers disclosed in the above international patent applications are the 3,11b-cis-dihydrotetrabenazines, so named because the hydrogen atoms at the 3 and 11b positions have a cis relative orientation.

There are four possible isomers of dihydrotetrabenazine having the 3,11b-cis configuration and these are the 2S,3S,11bR isomer, the 2R,3R,11bS isomer, the 2R,3S,11bR isomer and the 2S,3R,11bS isomer, which are as follows:

(a) the 2S,3S,11bR isomer of 3,11b-cis-dihydrotetrabenazine having the formula (Ia):

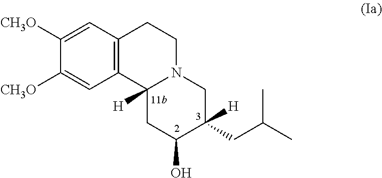

(b) the 2R,3R,11bS isomer of 3,11b-cis-dihydrotetrabenazine having the formula (Ib):

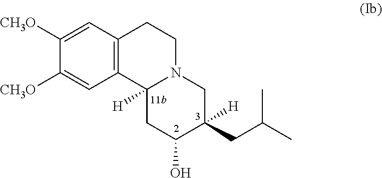

(c) the 2R,3S,11bR isomer of 3,11b-cis-dihydrotetrabenazine having the formula (Ic):

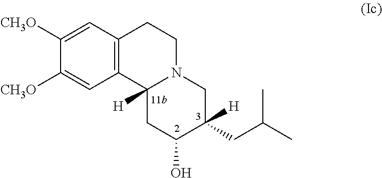

and
(d) the 2S,3R,11bS isomer of 3,11b-cis-dihydrotetrabenazine having the formula (Id):

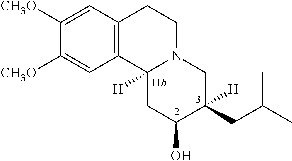

Isomer (Ib) above is disclosed in our earlier application WO 2007/017654 as being of benefit in treating cognitive deficit symptoms in schizophrenia.

In the above definitions and elsewhere herein, the stereochemistry of each isomer is defined using the “R and S” nomenclature developed by Cahn, Ingold and Prelog, see Advanced Organic Chemistry by Jerry March, 4^{th} Edition, John Wiley & Sons, New York, 1992, pages 109-114. In this patent application, the designations “R” or “S” are given in the order of the position numbers of the carbon atoms. Thus, for example, the 2R,3S,11bR isomer may be referred to in short hand form as RSR and so on.

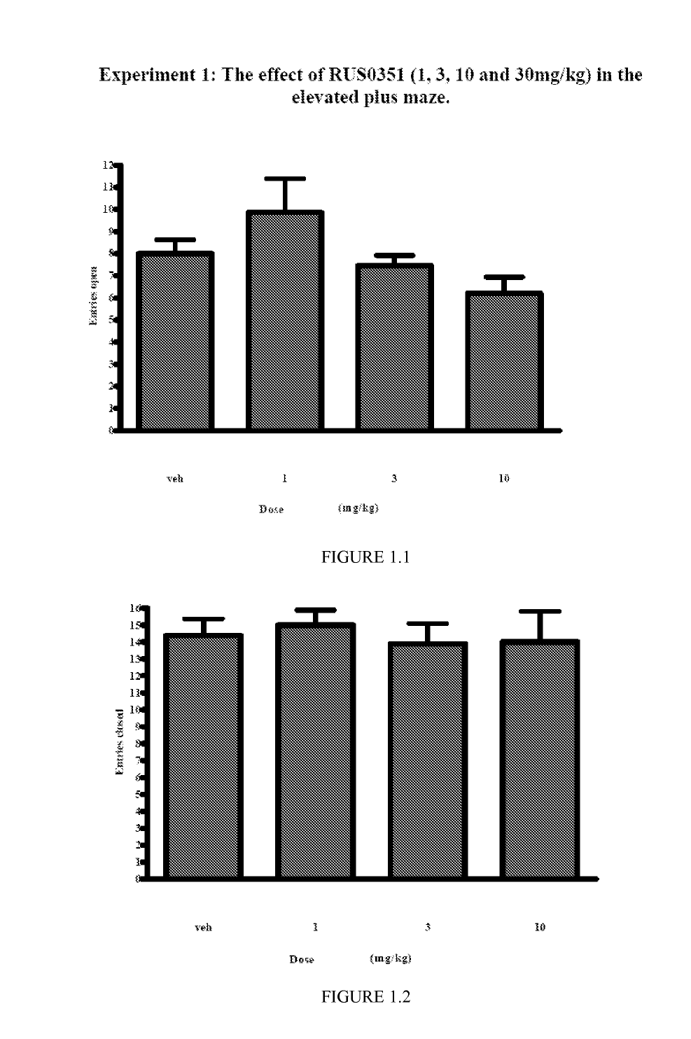

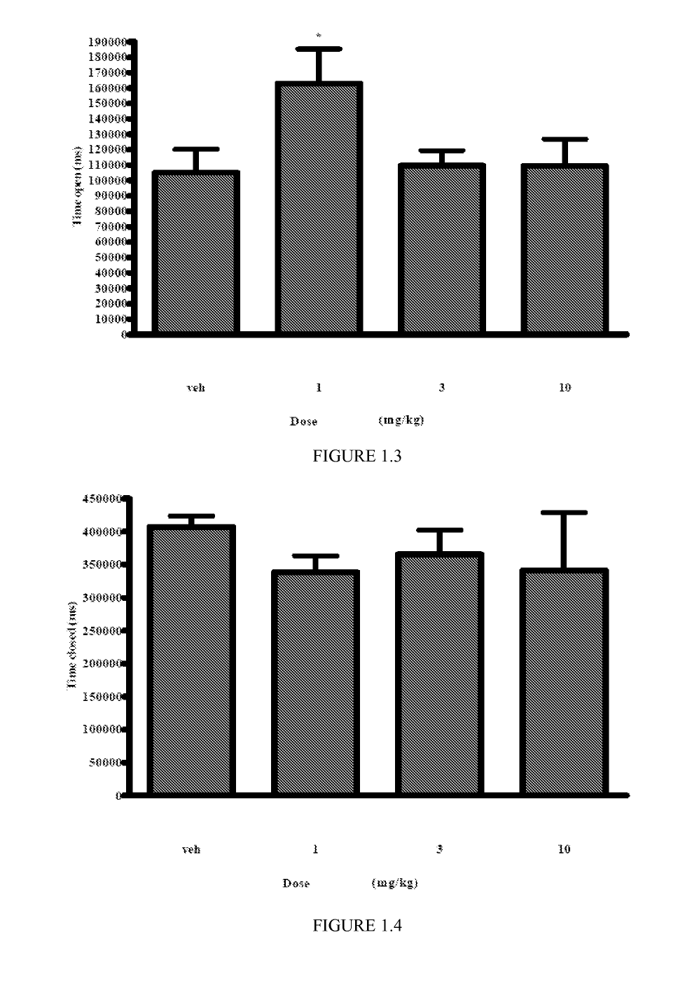

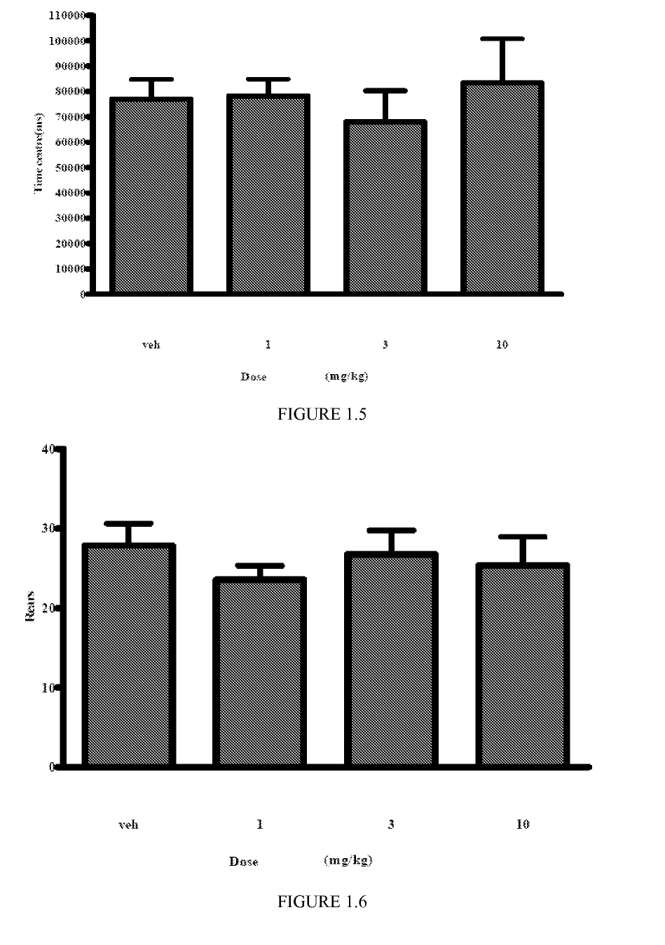

In addition to the 3,11b-cis-dihydrotetrabenazines, there are also four known 3,11b-trans-dihydrotetrabenazine isomers which have a trans relative orientation between the hydrogen atoms at the 3 and 11b positions) (see Kilbourn et al., Chirality, 9:59-62 (1997) and Brossi et al., Helv. Chim. Acta., vol. XLI, No. 193, pp 1793-1806 (1958). The four isomers are (+)-α-dihydrotetrabenazine, (−)-α-dihydrotetrabenazine, (+)-β-dihydrotetrabenazine and (−)-β-dihydrotetrabenazine. The structures of the four known trans-dihydrotetrabenazine isomers are considered to be as shown in FIG. 1.

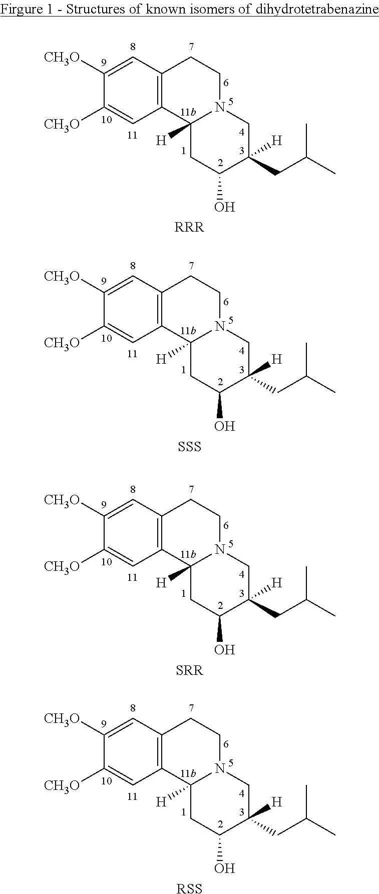

Some or all of the trans isomers are produced as metabolites of commercially available tetrabenazine and a dihydrotetrabenazine is believed to be primarily responsible for the activity of the drug (see Mehvar et al., Drug Metab. Disp, 15, 250-255 (1987) and J. Pharm. Sci., 76, No. 6, 461-465 (1987)).

### SUMMARY OF THE INVENTION

It has now been found that the 2R,3R,11bS isomer of 3,11b-cis-dihydrotetrabenazine of the formula (Ib) described in our earlier applications WO 2007/017654 and WO/2005/077946 exhibits biological properties which indicate that it should be useful in the prophylaxis or treatment of anxiety.

Accordingly, in a first aspect, the invention provides a 3,11b-cis-dihydrotetrabenazine of the formula (Ib):

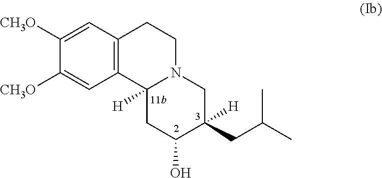

or a pharmaceutically acceptable salt thereof for use in the prophylaxis or treatment of anxiety.

The compound of formula (Ib), the 2R,3R,11bS isomer of 3,11b-cis-dihydrotetrabenazine, is also referred to herein as Isomer A.

The invention also provides:
The use of 3,11b-cis-dihydrotetrabenazine Isomer A for the manufacture of a medicament for the prophylaxis or treatment of anxiety.
3,11b-cis-dihydrotetrabenazine Isomer A for use in alleviating or preventing any one or more symptoms of anxiety.
A 3,11b-cis-dihydrotetrabenazine for use as defined herein wherein the anxiety is secondary to other psychiatric illness.
A 3,11b-cis-dihydrotetrabenazine for use as defined herein wherein the anxiety is primary anxiety.
A 3,11b-cis-dihydrotetrabenazine for use as defined herein wherein the anxiety is associated with a neurosis.
A 3,11b-cis-dihydrotetrabenazine for use as defined herein wherein the anxiety is phobic anxiety, for example wherein the phobic anxiety arises from social phobias, general phobias and specific phobias.
A 3,11b-cis-dihydrotetrabenazine for use as defined herein wherein the anxiety arises from an obsessional disorder.

The 3,11b-cis-dihydrotetrabenazine Isomer A may be used to prevent, stop or alleviate any one or more mental symptoms arising from or associated with anxiety such as anger, fear, apprehension, or worry, loss of patience, concentration difficulties, anxiety-related sleeping difficulties, anxiety-related depression, and/or obsessive behaviour development.

The 3,11b-cis-dihydrotetrabenazine Isomer A may be used to prevent, stop or alleviate any one or more physical symptoms arising form or associated with anxiety such as heart palpitations, pale skin, sweating, nausea, chest pain, shortness of breath, stomach aches, headache, excessive thirst, flatulence, diarrhoea, increased frequency of urination, sexual impotence, muscle pain, dizziness, pins and needles, tremors and painful or absent periods.

The cis-dihydrotetrabenazine Isomer A used in the invention may be in substantially pure form, for example at an isomeric purity of greater than 90%, typically greater than 95% and more preferably greater than 98%.

The term “isomeric purity” in the present context refers to the amount of 3,11b-cis-dihydrotetrabenazine Isomer A present relative to the total amount or concentration of dihydrotetrabenazine of all isomeric forms. For example, if 90% of the total dihydrotetrabenazine present in the composition is 3,11b-cis-dihydrotetrabenazine Isomer A, then the isomeric purity is 90%.

The 11b-cis-dihydrotetrabenazine Isomer A used in the invention may be in the form of a composition which is substantially free of 3,11b-trans-dihydrotetrabenazine, preferably containing less than 5% of 3,11b-trans-dihydrotetrabenazine, more preferably less than 3% of 3,11b-trans-dihydrotetrabenazine, and most preferably less than 1% of 3,11b-trans-dihydrotetrabenazine.

Isomer A can be characterised by its spectroscopic, optical and chromatographic properties, as described in the Examples below, and also by its absolute stereochemical configurations as determined by X-ray crystallography.

Isomer A may be presented in a substantially enantiomerically pure form or as a mixture with other 3,11b-cis-dihydrotetrabenazine enantiomers as described herein.

The terms “enantiomeric purity” and “enantiomerically pure” in the present context refer to the amount of 3,11b-cis-dihydrotetrabenazine Isomer A present relative to the total amount or concentration of dihydrotetrabenazine of all enantiomeric and isomeric forms. For example, if 90% of the total dihydrotetrabenazine present in the composition is in the form of a single enantiomer, then the enantiomeric purity is 90%.

By way of example, in each aspect and embodiment of the invention, Isomer A may be present in an enantiomeric purity of at least 55% (e.g. at least 60%, 65%, 70%, 75%, 80%, 85%, 90%, 95%, 97%, 98%, 99%, 99.5% or 100%).

Isomer A may also be presented in the form of mixtures with one or more of Isomers B (Isomer Ia), Isomer (Ic) and Isomer (Id) as defined herein. Such mixtures may be racemic mixtures or non-racemic mixtures. Examples of racemic mixtures include the racemic mixture of Isomer A and Isomer B (Isomer Ia).

### Pharmaceutically Acceptable Salts

Unless the context requires otherwise, a reference in this application to Isomer A includes within its scope not only the free base of the dihydrotetrabenazine but also its salts, and in particular acid addition salts.

Particular acids from which the acid addition salts are formed include acids having a pKa value of less than 3.5 and more usually less than 3. For example, the acid addition salts can be formed from an acid having a pKa in the range from +3.5 to −3.5.

Preferred acid addition salts include those formed with sulphonic acids such as methanesulphonic acid, ethanesulphonic acid, benzene sulphonic acid, toluene sulphonic acid, camphor sulphonic acid and naphthalene sulphonic acid.

One particular acid from which acid addition salts may be formed is methanesulphonic acid.

Acid addition salts can be prepared by the methods described herein or conventional chemical methods such as the methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free base form of the compound with the appropriate base or acid in water or in an organic solvent, or in a mixture of the two; generally, nonaqueous media such as ether, ethyl acetate, ethanol, isopropanol, or acetonitrile are used.

The methane sulphonic acid salt can be prepared as described below in the experimental section of this application.

The salts are typically pharmaceutically acceptable salts. However, salts that are not pharmaceutically acceptable may also be prepared as intermediate forms which may then be converted into pharmaceutically acceptable salts. Such non-pharmaceutically acceptable salt forms also form part of the invention.

### Methods for the Preparation of Dihydrotetrabenazine Isomers

The dihydrotetrabenazine of the invention can be prepared by a process comprising the reaction of a compound of the formula (II):

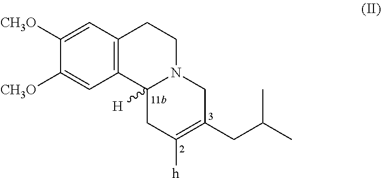

with a reagent or reagents suitable for hydrating the 2,3-double bond in the compound of formula (II) and thereafter where required separating and isolating the desired dihydrotetrabenazine isomer form.

The hydration of the 2,3-double bond can be carried out by hydroboration using a borane reagent such as diborane or a borane-ether (e.g. borane-tetrahydrofuran (THF)) to give an intermediate alkyl borane adduct followed by oxidation of the alkyl borane adduct and hydrolysis in the presence of a base. The hydroboration is typically carried out in a dry polar non-protic solvent such as an ether (e.g. THF), usually at a non-elevated temperature, for example room temperature. The borane-alkene adduct is typically oxidised with an oxidising agent such as hydrogen peroxide in the presence of a base providing a source of hydroxide ions, such as ammonium hydroxide or an alkali metal hydroxide, e.g. potassium hydroxide or sodium hydroxide. The hydroboration-oxidation-hydrolysis sequence of reactions of Process A typically provides dihydrotetrabenazine isomers in which the hydrogen atoms at the 2- and 3-positions have a trans relative orientation.

Compounds of the formula (II) can be prepared by reduction of tetrabenazine to give a dihydrotetrabenazine followed by dehydration of the dihydrotetrabenazine. Reduction of the tetrabenazine can be accomplished using an aluminium hydride reagent such as lithium aluminium hydride, or a borohydride reagent such as sodium borohydride, potassium borohydride or a borohydride derivative, for example an alkyl borohydride such as lithium tri-sec-butyl borohydride. Alternatively, the reduction step can be effected using catalytic hydrogenation, for example over a Raney nickel or platinum oxide catalyst. Suitable conditions for performing the reduction step are described in more detail below or can be found in U.S. Pat. No. 2,843,591 (Hoffmann-La Roche) and Brossi et al., Helv. Chim. Acta., vol. XLI, No. 193, pp 1793-1806 (1958).

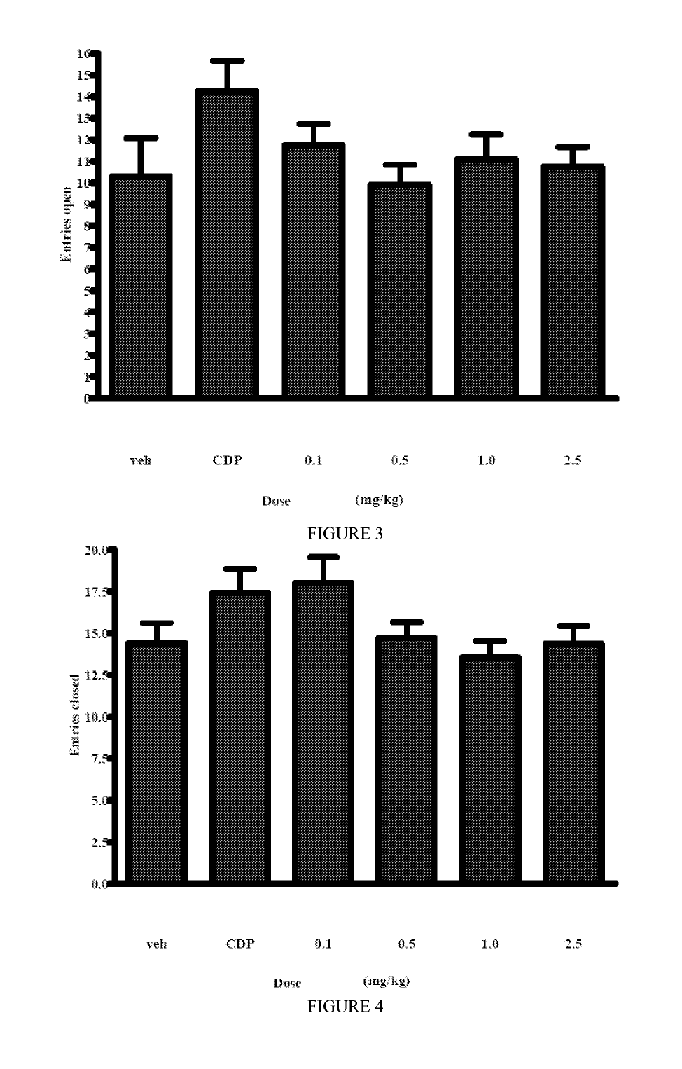

Because the tetrabenazine used as the starting material for the reduction reaction is typically a mixture of the RR and SS isomers (i.e. trans-tetrabenazine), the dihydrotetrabenazine formed by the reduction step will have the same trans configuration about the 3- and 11b positions and will take the form of one or more of the known dihydrotetrabenazine isomers shown in FIG. 3 above. Thus Process A may involve taking the known isomers of dihydrotetrabenazine, dehydrating them to form the alkene (II) and then “rehydrating” the alkene (II) using conditions that give the required cis dihydrotetrabenazine isomer of the invention.

Dehydration of the dihydrotetrabenazine to the alkene (II) can be carried out using a variety of standard conditions for dehydrating alcohols to form alkenes, see for example J. March (idem) pages 389-390 and references therein. Examples of such conditions include the use of phosphorus-based dehydrating agents such as phosphorus halides or phosphorus oxyhalides, e.g. POCl₃ and PCl₅. As an alternative to direct dehydration, the hydroxyl group of the dihydrotetrabenazine can be converted to a leaving group L such as halogen (e.g. chlorine or bromine) and then subjected to conditions (e.g. the presence of a base) for eliminating H-L. Conversion of the hydroxyl group to a halide can be achieved using methods well known to the skilled chemist, for example by reaction with carbon tetrachloride or carbon tetrabromide in the presence of a trialkyl or triaryl phosphine such as triphenyl phosphine or tributyl phosphine.

The tetrabenazine used as the starting material for the reduction to give the dihydrotetrabenazine can be obtained commercially or can be synthesised by the method described in U.S. Pat. No. 2,830,993 (Hoffmann-La Roche).

When the starting materials for process A above are mixtures of enantiomers, then the products of the processes will typically be pairs of enantiomers, for example racemic mixtures, possibly together with diastereoisomeric impurities. Unwanted diastereoisomers can be removed by techniques such as chromatography (e.g. HPLC) and the individual enantiomers can be separated by a variety of methods known to the skilled chemist. For example, they can be separated by means of:
(i) chiral chromatography (chromatography on a chiral support); or
(ii) forming a salt with an optically pure chiral acid, separating the salts of the two diastereoisomers by fractional crystallisation and then releasing the dihydrotetrabenazine from the salt; or
(iii) forming a derivative (such as an ester) with an optically pure chiral derivatising agent (e.g. esterifying agent), separating the resulting epimers (e.g. by chromatography) and then converting the derivative to the dihydrotetrabenazine.

One method of separating pairs of enantiomers obtained from Process A, and which has been found to be particularly effective, is to esterify the hydroxyl group of the dihydrotetrabenazine with an optically active form of Mosher's acid, such as the R (+) isomer shown below, or an active form thereof:

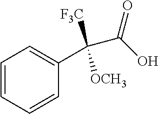

The resulting esters of the two enantiomers of the dihydrobenazine can then be separated by chromatography (e.g. HPLC) and the separated esters hydrolysed to give the individual dihydrobenazine isomers using a base such as an alkali metal hydroxide (e.g. NaOH) in a polar solvent such as methanol.

As an alternative to using mixtures of enantiomers as the starting materials in process A and then carrying out separation of enantiomers subsequently, process A can be carried out on single enantiomer starting materials leading to products in which the desired Isomer A predominates. Single enantiomers of the alkene (II) can be prepared by subjecting RR/SS tetrabenazine to a stereoselective reduction using lithium tri-sec-butyl borohydride to give a mixture of SRR and RSS enantiomers of dihydrotetrabenazine, separating the enantiomers (e.g. by fractional crystallisation) and then dehydrating a separated single enantiomer of dihydrotetrabenazine to give predominantly or exclusively a single enantiomer of the compound of formula (II).

Process A is illustrated in more detail below in Scheme 1.

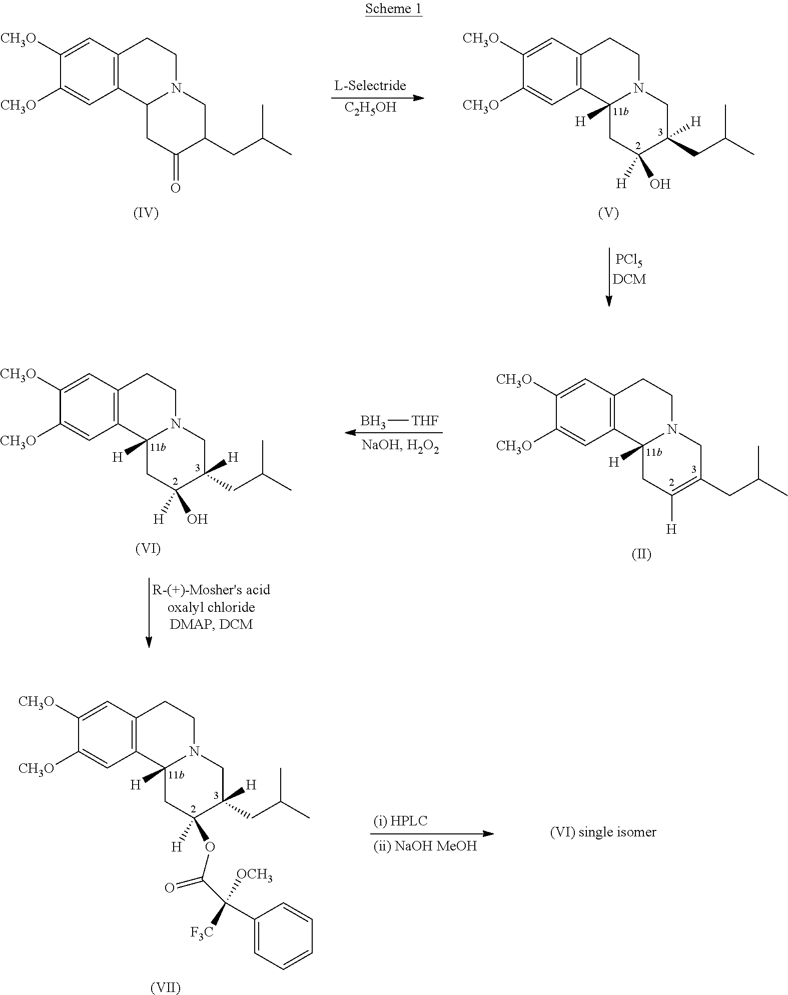

Scheme 1 illustrates the preparation of individual dihydrotetrabenazine isomers having the 2S,3S,11bR and 2R,3R,11bS configurations in which the hydrogen atoms attached to the 2- and 3-positions are arranged in a trans relative orientation. This reaction scheme includes Process A defined above.

The starting point for the sequence of reactions in Scheme 1 is commercially available tetrabenazine (IV) which is a racemic mixture of the RR and SS optical isomers of tetrabenazine. In each of the RR and SS isomers, the hydrogen atoms at the 3- and 11b-positions are arranged in a trans relative orientation. As an alternative to using the commercially available compound, tetrabenazine can be synthesised according to the procedure described in U.S. Pat. No. 2,830,993 (see in particular example 11).

The racemic mixture of RR and SS tetrabenazine is reduced using the borohydride reducing agent lithium tri-sec-butyl borohydride (“L-Selectride”) to give a mixture of the known 2S,3R,11bR and 2R,3S,11bS isomers (V) of dihydrotetrabenazine, of which only the 2S,3R,11bR isomer is shown for simplicity. By using the more sterically demanding L-Selectride as the borohydride reducing agent rather than sodium borohydride, formation of the RRR and SSS isomers of dihydro-tetrabenazine is minimised or suppressed.

The dihydrotetrabenazine isomers (V) are reacted with a dehydrating agent such as phosphorus pentachloride in a non-protic solvent such as a chlorinated hydrocarbon (for example chloroform or dichloromethane, preferably dichloromethane) to form the unsaturated compound (II) as a pair of enantiomers, only the R-enantiomer of which is shown in the Scheme. The dehydration reaction is typically carried out at a temperature lower than room temperature, for example at around 0-5° C.

The unsaturated compound (II) is then subjected to a stereoselective re-hydration to generate the dihydrotetrabenazine (VI) and its mirror image or antipode (not shown) in which the hydrogen atoms at the 3- and 11b-positions are arranged in a cis relative orientation and the hydrogen atoms at the 2- and 3-positions are arranged in a trans relative orientation. The stereoselective rehydration is accomplished by a hydroboration procedure using borane-THF in tetrahydrofuran (THF) to form an intermediate borane complex (not shown) which is then oxidised with hydrogen peroxide in the presence of a base such as sodium hydroxide.

An initial purification step may then be carried out (e.g. by HPLC) to give the product (V) of the rehydration reaction sequence as a mixture of the 2S,3S,11bR and 2R,3R,11bS isomers of which only the 2S,3S,11bR isomer is shown in the Scheme. In order to separate the isomers, the mixture is treated with R (+) Mosher's acid, in the presence of oxalyl chloride and dimethylaminopyridine (DMAP) in dichloromethane to give a pair of diastereoisomeric esters (VII) (of which only one diastereoisomer is shown) which can then be separated using HPLC. The individual esters can then be hydrolysed using an alkali metal hydroxide such as sodium hydroxide to give a single isomer (VI).

In a variation of the sequence of steps shown in Scheme 1, following the reduction of RR/SS tetrabenazine, the resulting mixture of enantiomers of the dihydrotetrabenazine (V) can be separated to give the desired individual enantiomer. Separation can be carried out by forming a salt with a chiral acid such as (+) or (−) camphorsulphonic acid, separating the resulting diastereoisomers by fractional crystallisation to give a salt of a single enantiomer and then releasing the free base from the salt.

The separated dihydrotetrabenazine enantiomer can be dehydrated to give a single enantiomer of the alkene (II). Subsequent rehydration of the alkene (II) will then give predominantly or exclusively a single enantiomer of the cis-dihydrotetrabenazine (VI). An advantage of this variation is that it does not involve the formation of Mosher's acid esters and therefore avoids the chromatographic separation typically used to separate Mosher's acid esters.

### Pharmaceutical Formulations

The cis-dihydrotetrabenazine compound of the invention is typically administered in the form of a pharmaceutical composition.

The pharmaceutical compositions can be in any form suitable for oral, parenteral, topical, intranasal, intrabronchial, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, sprays, powders, granules, elixirs and suspensions, sublingual tablets, sprays, wafers or patches and buccal patches.

Pharmaceutical compositions containing the dihydrotetrabenazine compound of the invention can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, talc, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped mouldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compound of the invention will generally be presented in unit dosage form and, as such, will typically contain sufficient compound to provide a desired level of biological activity. For example, a formulation intended for oral administration may contain from 2 milligrams to 200 milligrams of active ingredient, more usually from 10 milligrams to 100 milligrams, for example, 12.5 milligrams, 25 milligrams and 50 milligrams.

### Methods of Treatment

The active compound will be administered to a patient in need thereof (for example a human or animal patient) in an amount sufficient to achieve the desired therapeutic effect.

The patient in need of such administration is a patient suffering from or exhibiting, or at risk of suffering from or exhibiting, one or more characteristic of anxiety.

The desired effect can be the prevention, alleviation or reduction of the severity of anxiety or one or more symptoms thereof. Such symptoms are well known to the skilled person (e.g. a skilled physician) who will be able to judge through clinical evaluation and testing in a conventional manner whether or not the administration of a compound of the invention has resulted in a change in the symptoms exhibited by the patient.

The compound will typically be administered in amounts that are therapeutically or prophylactically useful and which generally are non-toxic. However, in certain situations, the benefits of administering the dihydrotetrabenazine compound of the invention may outweigh the disadvantages of any toxic effects or side effects, in which case it may be considered desirable to administer compounds in amounts that are associated with a degree of toxicity.

A typical daily dose of the compound can be up to 1000 mg per day, for example in the range from 0.01 milligrams to 10 milligrams per kilogram of body weight, more usually from 0.025 milligrams to 5 milligrams per kilogram of body weight, for example up to 3 milligrams per kilogram of bodyweight, and more typically 0.15 milligrams to 5 milligrams per kilogram of bodyweight although higher or lower doses may be administered where required.

Ultimately, however, the quantity of compound administered will be commensurate with the nature of the disease or physiological condition being treated and the therapeutic benefits and the presence or absence of side effects produced by a given dosage regimen, and will be at the discretion of the physician.

### EXAMPLES

The following non-limiting examples illustrate the synthesis and properties of the 3,11b-cis-dihydrotetrabenazine isomers. The examples describe all four isomers of 3,11b-cis-dihydrotetrabenazine although the invention is limited to the therapeutic uses of Isomer A (the compound of formula (Ib)). The examples relating to the other isomers are retained as comparative examples.

### Example 1

### Preparation of 2S,3S,11bR and 2R,3R,11bS Isomers of Dihydrotetrabenazine

### 1A. Reduction of RR/SS Tetrabenazine

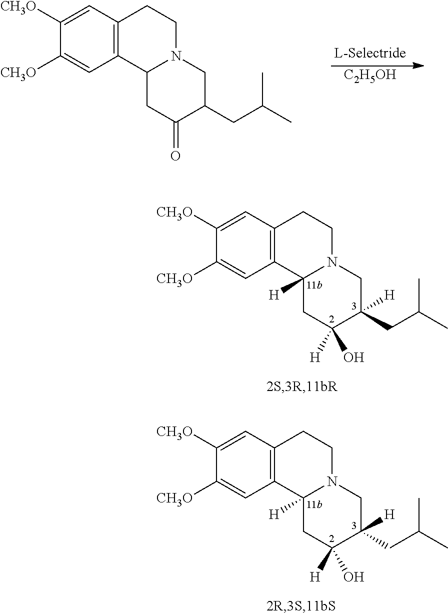

1M L-Selectride® in tetrahydrofuran (135 ml, 135 mmol, 2.87 eq.) was added slowly over 30 minutes to a stirred solution of tetrabenazine RR/SS racemate (15 g, 47 mmol) in ethanol (75 ml) and tetrahydrofuran (75 ml) at 0° C. After addition was complete the mixture was stirred at 0° C. for 30 minutes and then allowed to warm to room temperature.

The mixture was poured onto crushed ice (300 g) and water (100 ml) added. The solution was extracted with diethyl ether (2×200 ml) and the combined ethereal extracts washed with water (100 ml) and partly dried over anhydrous potassium carbonate. Drying was completed using anhydrous magnesium sulphate and, after filtration, the solvent was removed at reduced pressure (shielded from the light, bath temperature <20° C.) to afford a pale yellow solid.

The solid was slurried with petroleum ether (30-40° C.) and filtered to afford a white powdery solid (12 g, 80%).

1B. Dehydration of reduced Tetrabenazine

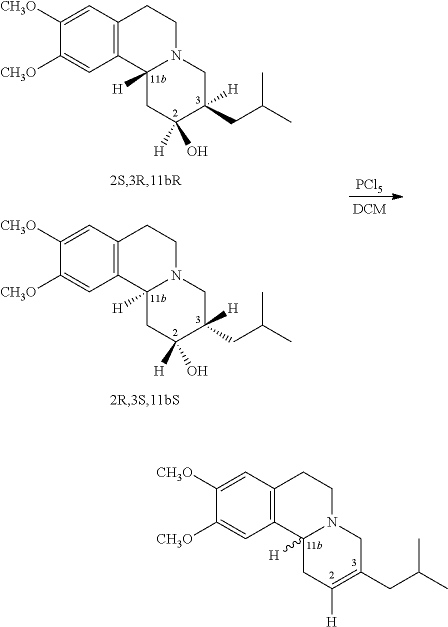

Phosphorous pentachloride (32.8 g, 157.5 mmol, 2.5 eq) was added in portions over 30 minutes to a stirred solution of the reduced tetrabenazine product from Example 1A (20 g, 62.7 mmol) in dichloromethane (200 ml) at 0° C. After the addition was complete, the reaction mixture was stirred at 0° C. for a further 30 minutes and the solution poured slowly into 2M aqueous sodium carbonate solution containing crushed ice (0° C.). Once the initial acid gas evolution had ceased the mixture was basified (ca. pH 12) using solid sodium carbonate.

The alkaline solution was extracted using ethyl acetate (800 ml) and the combined organic extracts dried over anhydrous magnesium sulphate. After filtration the solvent was removed at reduced pressure to afford a brown oil, which was purified by column chromatography (silica, ethyl acetate) to afford the semi-pure alkene as a yellow solid (10.87 g, 58%).

1C. Hydration of the Crude Alkene from Example 1B

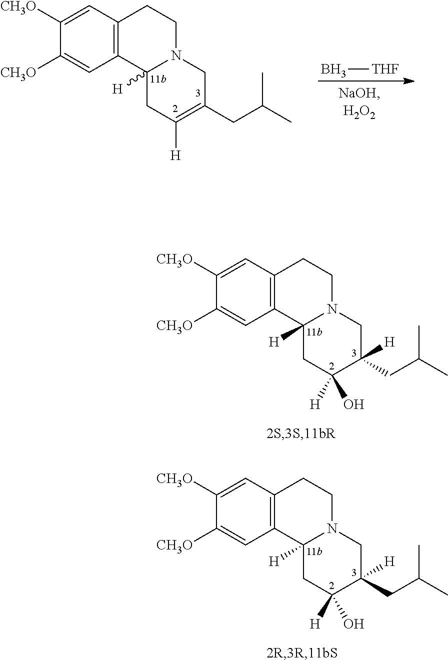

A solution of the crude alkene (10.87 g, 36.11 mmol) from Example 1B in dry THF (52 ml) at room temperature was treated with 1M borane-THF (155.6 ml, 155.6 mmol, 4.30 eq) added in a dropwise manner. The reaction was stirred for 2 hours, water (20 ml) was added and the solution basified to pH 12 with 30% aqueous sodium hydroxide solution.

Aqueous 30% hydrogen peroxide solution (30 ml) was added to the stirred alkaline reaction mixture and the solution was heated to reflux for 1 hour before being allowed to cool. Water (100 ml) was added and the mixture extracted with ethyl acetate (3×250 ml). The organic extracts were combined and dried over anhydrous magnesium sulphate and after filtration the solvent was removed at reduced pressure to afford a yellow oil (9 g).

The oil was purified using preparative HPLC (Column: Lichrospher Si60, 5 μm, 250×21.20 mm, mobile phase: hexane:ethanol:dichloromethane (85:15:5); UV 254 nm, flow: 10 ml min⁻¹) at 350 mg per injection followed by concentration of the fractions of interest under vacuum. The product oil was then dissolved in ether and concentrated once more under vacuum to give the dihydrotetrabenazine racemate shown above as a yellow foam (5.76 g, 50%).

### 1D. Preparation of Mosher's Ester Derivatives

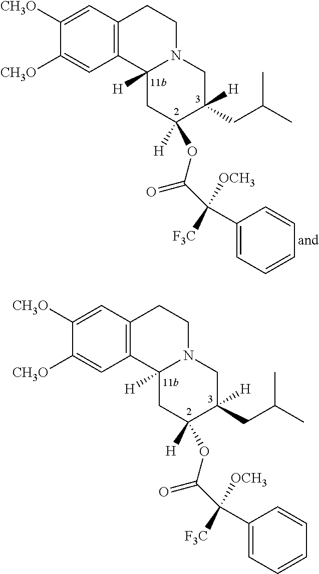

R-(+)-α-methoxy-α-trifluoromethylphenyl acetic acid (5 g, 21.35 mmol), oxalyl chloride (2.02 ml) and DMF (0.16 ml) were added to anhydrous dichloromethane (50 ml) and the solution was stirred at room temperature for 45 minutes. The solution was concentrated under reduced pressure and the residue was taken up in anhydrous dichloromethane (50 ml) once more. The resulting solution was cooled using an ice-water bath and dimethylaminopyridine (3.83 g, 31.34 mmol) was added followed by a pre-dried solution (over 4A sieves) in anhydrous dichloromethane of the solid product of Example 1C (5 g, 15.6 mmol). After stirring at room temperature for 45 minutes, water (234 ml) was added and the mixture extracted with ether (2×200 ml). The ether extract was dried over anhydrous magnesium sulphate, passed through a pad of silica and the product eluted using ether.

The collected ether eluate was concentrated under reduced pressure to afford an oil which was purified using column chromatography (silica, hexane:ether (10:1)).

Evaporation of the collected column fractions of interest and removal of the solvent at reduced pressure gave a solid which was further purified using column chromatography (silica, hexane:ethyl acetate (1:1)) to give three main components which were partially resolved into Mosher's ester peaks 1 and 2.

Preparative HPLC of the three components (Column: 2×Lichrospher Si60, 5 μm, 250×21.20 mm, mobile phase: hexane:isopropanol (97:3), UV 254 nm; flow: 10 ml min⁻¹) at 300 mg loading followed by concentration of the fractions of interest under vacuum gave the pure Mosher's ester derivatives

### Peak 1 (3.89 g, 46.5%)

### Peak 2 (2.78 g, 33%)

The fractions corresponding to the two peaks were subjected to hydrolysis to liberate the individual dihydrotetrabenazine isomers identified and characterised as Isomers A and B. Isomers A and B are each believed to have one of the following structures

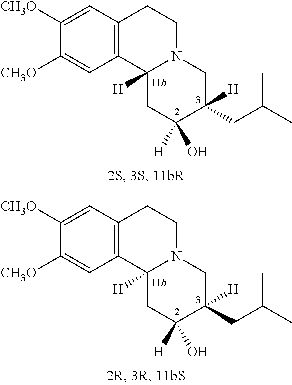

More specifically, Isomer B is believed to have the 2S, 3S,11bR absolute configuration on the basis of the X-ray crystallography experiments described in our earlier International patent applications WO/2007/017654, WO/2007/017643, WO/2007/007105, see in particular Example 4 in WO/2007/017654. Since Isomer A is the antipode of Isomer B, Isomer A must have the 2R,3R,11bS configuration.

### 1E. Hydrolysis of Peak 1 to Give Isomer A

Aqueous 20% sodium hydroxide solution (87.5 ml) was added to a solution of Mosher's ester peak 1 (3.89 g, 7.27 mmol) in methanol (260 ml) and the mixture stirred and heated to reflux for 150 minutes. After cooling to room temperature water (200 ml) was added and the solution extracted with ether (600 ml), dried over anhydrous magnesium sulphate and after filtration, concentrated under reduced pressure.

The residue was dissolved using ethyl acetate (200 ml), the solution washed with water (2×50 ml), the organic phase dried over anhydrous magnesium sulphate and after filtration, concentrated under reduced pressure to give a yellow foam. This material was purified by column chromatography (silica, gradient elution of ethyl acetate:hexane (1:1) to ethyl acetate). The fractions of interest were combined and the solvent removed at reduced pressure. The residue was taken up in ether and the solvent removed at reduced pressure once more to give Isomer A as an off-white foam (1.1 g, 47%).

Isomer A, which is believed to have the 2R,3R,11bS configuration (the absolute stereochemistry was not determined), was characterized by ¹H-NMR, ¹³C-NMR, IR, mass spectrometry, chiral HPLC and ORD. The IR, NMR and MS data for isomer A are set out in Table 1 and the Chiral HPLC and ORD data are set out in Table 3.

### 1F. Hydrolysis of Peak 2 to Give Isomer B

Aqueous 20% sodium hydroxide solution (62.5 ml) was added to a solution of Mosher's ester peak 2 (2.78 g, 5.19 mmol) in methanol (185 ml) and the mixture stirred and heated to reflux for 150 minutes. After cooling to room temperature water (142 ml) was added and the solution extracted with ether (440 ml), dried over anhydrous magnesium sulphate and after filtration, concentrated under reduced pressure.

The residue was dissolved using ethyl acetate (200 ml), the solution washed with water (2×50 ml), the organic phase dried over anhydrous magnesium sulphate and after filtration, concentrated under reduced pressure. Petroleum ether (30-40° C.) was added to the residue and the solution concentrated under vacuum once more to give Isomer B as a white foam (1.34 g, 81%).

Isomer B, which is believed to have the 2S,3S,11bR configuration, was characterized by ¹H-NMR, ¹³C-NMR, IR, mass spectrometry, chiral HPLC, ORD and X-ray crystallography. The IR, NMR and MS data for Isomer B are set out in Table 1 and the Chiral HPLC and ORD data are set out in Table 3. The X-ray crystallography data are set out in Example 4 in WO/2007/017654.

TABLE 1
Spectroscopic Data

| | ¹H-NMR | ¹³C-NMR | IR | Mass |
|---|---|---|---|---|
| | spectrum | spectrum | Spectrum | Spectrum |
| Dihydrotetrabenazine isomer | (CDCl₃) | (CDCl₃) | (KBr solid) | (ES⁺) |
| 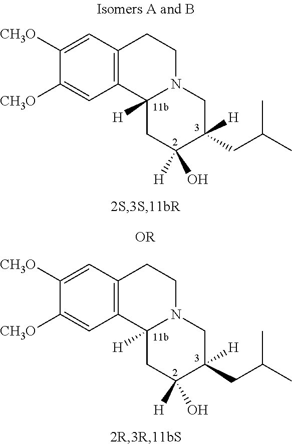 | 6.67δ 1H (s); 6.57δ 1H (s); 3.84δ 6H (s); 3.55δ 1H (br. d); 3.08δ 1H (m); 2.79δ 2H (m); 2.55δ 3H (m); 2.17δ 1H (m); 1.72δ 6H (m); 1.02δ 1H (m); 0.88δ 6H (t) | 147.7δ; 147.6δ; 130.5δ; 127.6δ; 112.1δ; 108.4δ; 70.5δ; 57.5δ; 56.5δ; 56.3δ; 54.8δ; 53.2δ; 40.4δ; 40.1δ; 36.0δ; 28.8δ; 26.2δ; 23.7δ; 22.9δ | 2950 cm⁻¹; 2928 cm⁻¹; 2868 cm⁻¹; 2834 cm⁻¹; 1610 cm⁻¹; 1511 cm⁻¹; 1464 cm⁻¹; 1364 cm⁻¹; 1324 cm⁻¹; 1258 cm⁻¹; 1223 cm⁻¹; 1208 cm⁻¹; 1144 cm⁻¹; 1045 cm⁻¹; 1006 cm⁻¹; 870 cm⁻¹; 785 cm⁻¹; 764 cm⁻¹ | MH⁺ 320 |

TABLE 2
Chromatography and ORD Data

| | Chiral HPLC Methods and | ORD |
|---|---|---|
| Dihydrotetrabenazine isomer | Retention Times | (MeOH, 21° C.) |
| 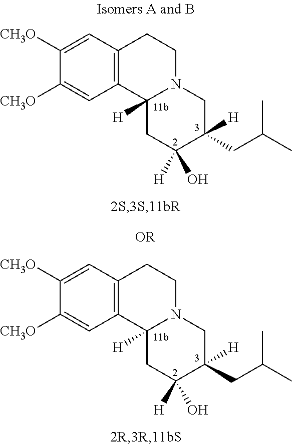 | Column: Chirex (S)-VAL, (R)-NEA, 250 × 4.6 mm Mobile phase: Hexane:1,2-dichloroethane:ethanol (36:62:2) Flow: 1.0 ml min⁻¹ UV: 254 nm Retention times: Isomer A 16.6 min Isomer B 15.3 min | Isomer A [α_{D}] −114.6° Isomer B [α_{D}] +123° |

### Example 2

### Preparation of the Mesylate Salt of Isomer A

The methanesulphonate salt of Isomer A can be prepared by dissolving a mixture of 1 equivalent of Isomer A and 1 equivalent of methane sulphonic acid in the minimum amount of ethanol and then adding diethyl ether. The resulting white precipitate that forms is collected by filtration and dried in vacuo to give the mesylate salt.

### Example 3

### Assessment of the Anxiolytic Properties of Isomer A by the Elevated Plus Maze Paradigm

The elevated plus maze has been widely used as a test for anxiety, as it avoids confounding effects on consummatory responses or sensitivity to shock and it has a certain level of ethological relevance (see Rodgers R J, Cole J C (1994) The elevated plus maze. Pharmacological methods and ethology. In: Cooper, S J, Hendrie C A (eds) Ethology and Psychopharmacology. J. Wiley & Sons Ltd, pp 9-941994).

The maze consists of opposite pairs of open and closed arms, and the proportion of exploration carried out on the open arms is taken to be a measure of anxiety. Thus the percentage of open arm entries is increased by known anxiolytic agents and reduced by anxiogenic compounds (Pellow, S, Chopin P, File S E, Briley, M (1985) Validation of open: closed arm entries in an elevated plus maze as a measure of anxiety in the rat. J. Neurosci. Meth. 14: 149-167). In addition, the plus maze may be used to examine the behavioural response to stress, as exposure induces the release of corticosterone File, S E, Pellow, S (1987) Behavioural pharmacology of minor tranquillisers. J. Pharmacol. Exp. Ther. 35: 265-290). The elevated plus maze is based on an approach conflict model and has been validated as a model of anxiety in the rat (Tomkins D M (1990) The behavioural effects of anxiogenic agents in rodents. PhD thesis. Postgraduate studies in Pharmacology. University of Bradford).

Two studies were carried out using the elevated plus maze method and the aim of the studies was to assess the ability of a Isomer A for its ability to produce an anxiolytic effect in female hooded-Lister rats. The working hypothesis was that a low dose range of Isomer A would produce an anxiolytic effect comparable to that produced by the benzodiazepine, chlordiazepoxide.

### Methods

### Study 1

### Subjects:

A total of 90 female hooded-Lister rats were used as subjects for study 1. Female hooded-Lister rats (Harlan, UK) were obtained as adults and weighed approximately 200-220 g at the start of the study. Subjects were housed in routinely used home cages in groups of 5 per cage. Home cages measured 59×35×24 cm. Rats were maintained under standard laboratory conditions on a 12:12 hour light/dark cycle (lights on at 08:00 h). Experimental procedures were performed in the light phase. Laboratory rooms were temperature controlled at 21±2° C. and humidity remained at 40-50% throughout the study. Rats were tested in the naïve state i.e. they had no exposure to the plus maze prior to testing, although they were handled and received sham injections prior to testing.

### Drugs:

Isomer A (1.0-30.0 mg/kg) and chlordiazepoxide (1.0-5.0 mg/kg) were tested. Isomer A was dissolved in water and administered via the oral route 30 minutes prior to testing. Chlordiazepoxide (CDP) was dissolved in saline and administered via the intraperitoneal route 30 min prior to testing.

### Study 2

### Subjects:

A total of 60 female hooded-Lister rats were used as subjects for study B. Female hooded-Lister rats (Charles River, UK) were obtained as adults and weighed approximately 220-250 g at the start of the study. Subjects were housed in routinely used home cages in groups of 5 per cage. Home cages measured 59×35×24 cm. Rats were maintained under standard laboratory conditions on a 12:12 hour light/dark cycle (lights on at 08:00 h). Experimental procedures were performed in the light phase. Laboratory rooms were temperature controlled at 21±2° C. and humidity remained at 40-50% throughout the study. Rats were tested in the naïve state i.e. they had no exposure to the plus maze prior to testing, although they were handled and received sham injections prior to testing.

### Drugs:

Isomer A (0.1-2.5 mg/kg) and chlordiazepoxide (2.5 mg/kg) were tested. Isomer A was dissolved in water and administered via the oral route (p.o.) 30 minutes prior to testing. Chlordiazepoxide (CDP) was dissolved in saline and administered via the intraperitoneal route (i.p.) 30 min prior to testing.

### Behaviour on the Elevated Plus Maze (Study 1 and Study 2)

The maze consisted of a +shaped maze, made of Perspex, with two open arms (30×5 cm) and two closed arms (30×5 cm). The maze was arranged so that like arms are opposing. The closed arms were surrounded by matt black walls on all sides (10 cm in height). The maze was elevated to a height of 50 cm, and the surface was covered with a removable rubber matting. The behaviour of drug or vehicle-treated rats was monitored by means of a video camera attached to a TV monitor and video recorder. The rat was placed in the centre square and behaviour monitored over a 10 min test period. Any animal which did not remain on the maze for the entire experimental period was excluded from subsequent analysis. The maze was cleaned between tests and rats were placed on the maze in a randomised manner in order to minimise the effects of familiar cage mate odours.

Behaviour was recorded and the following parameters were analysed from the video tapes using the Hindsight® program for ethological analysis of behaviour: time spent on, and entries into, the open and closed arms, time spent in the centre square and rearing on the open and closed arms.

### Statistical Analysis (Studies 1 and 2)

Elevated plus maze data were analysed by a one way ANOVA, followed by Dunnett's t test to assess drug effects on number of entries into the open and closed arms, length of time spent, and number of rears in, open and closed arms, and time spent in the centre square compared with vehicle.

### Results

### Study 1

The results obtained from Study 1 are illustrated in FIGS. 1.1 to 2.6.

FIG. 1.1 illustrates the effect of Isomer A treatment (1.0-10 mg/kg, p.o.) on total open arm entries in a 10 minute trial on the elevated plus-maze. Data are expressed as mean±s.e.m (n=5-9 per group). The data illustrate that there was a significant effect of Isomer A on total entries into the open arms of the elevated plus maze [F(3,28)=2.43, p<0.05]. A trend towards an increase in open arm entries was observed at 1 mg/kg of Isomer A.

FIG. 1.2. illustrates the effect of Isomer A treatment (1.0-10 mg/kg, p.o.) on total closed arm entries in a 10 minute trial on the elevated plus-maze. Data are expressed as mean±s.e.m. (n=5-9 per group) and were analysed by ANOVA and post-hoc Dunnett's t-test. The data demonstrate that there was no significant effect of Isomer A on total entries into the closed arms of the elevated plus maze [F(3,28)=0.17, NS].

FIG. 1.3 illustrates the effect of Isomer A treatment (1.0-10 mg/kg, p.o.) on time spent on the open arms in a 10 minute trial on the elevated plus-maze. Data are expressed as mean±s.e.m (n=5-9 per group) and were analysed by ANOVA and post-hoc Dunnett's t-test. (*P<0.05; significant increase in time spent on the open arms compared to vehicle). The data illustrate that there was an overall significant effect of Isomer A on time spent on the open arms of the elevated plus maze [F(3,28)=2.87, p<0.05]. Post-hoc analysis revealed that at 1 mg/kg of Isomer A, there was a significant increase in time spent on the open arms of the elevated plus maze (p<0.05).

FIG. 1.4 illustrates the effect of Isomer A treatment (1.0-10 mg/kg, p.o.) on time spent on the closed arms in a 10 min trial on the elevated plus-maze. Data are expressed as mean±s.e.m (n=5-9 per group) and were analysed by ANOVA and post-hoc Dunnett's t-test. The data demonstrate that there was no significant effect of Isomer A on time spent on the closed arms of the elevated plus maze [F(3,28)=0.62, NS] FIG. 1.4. Isomer A showed a trend towards a reduction in time spent on the closed arm (most marked at 1.0 mg/kg) but this effect failed to reach statistical significance.

FIG. 1.5 illustrates the effect of Isomer A treatment (1.0-10 mg/kg, p.o.) on time spent in the centre square in a 10 min trial on the elevated plus-maze. Data are expressed as mean±s.e.m (n=5-9 per group) and were analysed by ANOVA and post-hoc Dunnett's t-test. The data demonstrate that there was no significant effect of Isomer A on time spent in the centre square of the elevated plus maze [F(3,28)=0.84, NS].

FIG. 1.6 illustrates the effect of Isomer A treatment (1.0-10 mg/kg, p.o.) on number of rears in a 10 minute trial on the elevated plus-maze. Data are expressed as mean±s.e.m (n=5-9 per group) and were analysed by ANOVA and post-hoc Dunnett's t-test. The data demonstrate that there was no significant effect of Isomer A on the number of rears on the elevated plus maze [F(3,28)=0.44, NS]. At 30 mg/kg of Isomer A, the animals did not behave normally on the elevated plus maze, their overall activity levels were markedly reduced, and they remained stationary for much of the 10 minute test period. Hence, behaviour was not scored and no data are shown.

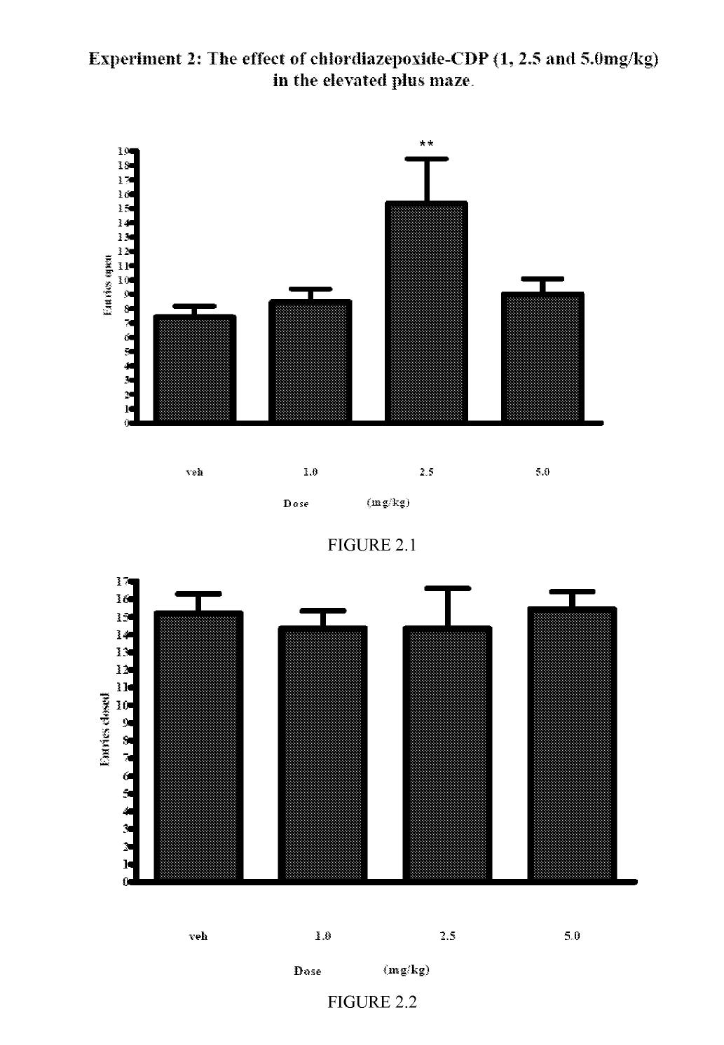

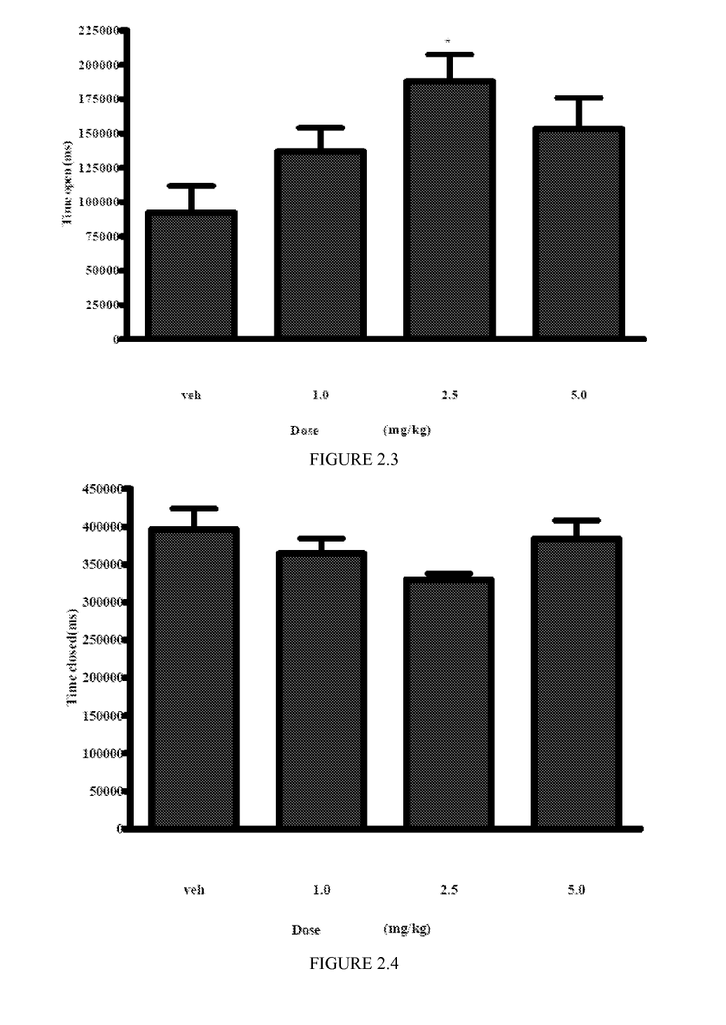

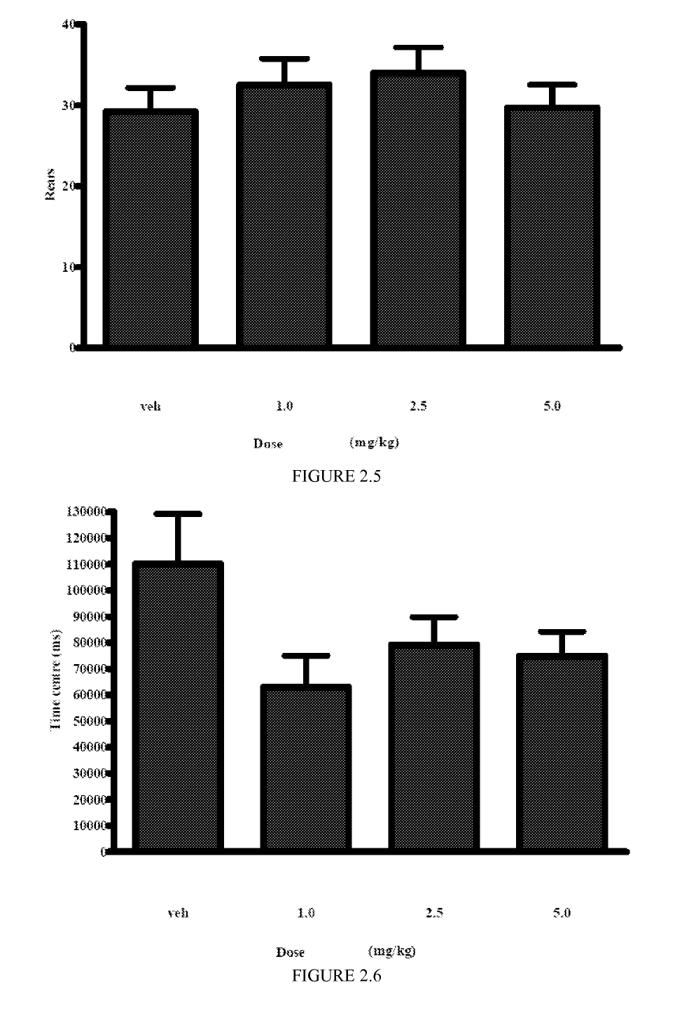

FIG. 2.1 illustrates the effect of chlordiazepoxide-CDP-treatment (1.0-5.0 mg/kg, i.p.) on total open arm entries in a 10 minute trial on the elevated plus-maze. Data are expressed as mean±s.e.m. (n=7-10 per group) and were analysed by ANOVA and post-hoc Dunnett's t-test (**P<0.01; significant increase in open arm entries compared to vehicle). The data demonstrate that there was a significant effect of CDP on total entries into the open arms of the elevated plus maze [F(3,34)=4.2, p<0.05]. Post-hoc analysis revealed that 2.5 mg/kg of CDP significantly increased the number of entries into the open arms of the elevated plus maze (p<0.01).

FIG. 2.2 illustrates the effect of chlordiazepoxide treatment-CDP (1.0-5.0 mg/kg, i.p.) on total closed arm entries in a 10 minute trial on the elevated plus-maze. Data are expressed as mean±s.e.m (n=7-10 per group) and were analysed by ANOVA and post-hoc Dunnett's t-test. The data demonstrate that there was no significant effect of CDP on total entries into the closed arms of the elevated plus maze [F(3,34)=0.14, NS].

FIG. 2.3 illustrates the effect of chlordiazepoxide-CDP-treatment (1.0-5.0 mg/kg, i.p) on time spent on the open arms in a 10 min trial on the elevated plus-maze. Data are expressed as mean±s.e.m (n=7-10 per group) and were analysed by ANOVA and post-hoc Dunnett's t-test. (*P<0.05; significant increase in time spent on the open arms compared with vehicle). The data demonstrate that there was an overall significant effect of CDP on time spent on the open arms of the elevated plus maze [F(3,34)=4.43, p<0.05]. All three doses of CDP increased time spent on the open arms, post-hoc analysis of the data revealed that this effect reached statistical significance at 2.5 mg/kg (p<0.05).

FIG. 2.4 illustrates the effect of chlordiazepoxide-CDP-treatment (1.0-5.0 mg/kg, i.p.) on time spent on the closed arms in a 10 min trial on the elevated plus-maze. Data are expressed as mean±s.e.m (n=7-10 per group) and were analysed by ANOVA and post-hoc Dunnett's t-test. The data demonstrate that there was no significant effect of CDP on time spent on the closed arms of the elevated plus maze [F(3,34)=1.85, NS]. However, post-hoc analysis of these data revealed that the effect of 2.5 mg/kg of CDP to reduce time spent on the closed arms approached significance (p=0.08).

FIG. 2.5 illustrates the effect of chlordiazepoxide-CDP— treatment (1.0-5.0 mg/kg, i.p.) on number of rears in a 10 min trial on the elevated plus-maze. Data are expressed as mean±s.e.m. (n=7-10 per group) and were analysed by ANOVA and post-hoc Dunnett's t-test. The data demonstrate that CDP had no significant effect on the number of rears on the elevated plus maze [F(3.34)=0.56, NS].

FIG. 2.6 illustrates the effect of chlordiazepoxide treatment (1.0-5.0 mg/kg, i.p.) on time spent in the centre square in a 10 minute trial on the elevated plus-maze. Data are expressed as mean±s.e.m (n=7-10 per group) and were analysed by ANOVA and post-hoc Dunnett's t-test. The data demonstrate that CDP did not produce a significant effect on time spent in the centre square of the elevated plus maze [F(3, 34)=2.15, NS]. However, post-hoc analysis of the data revealed that the effect of 1.0 mg/kg of CDP to reduce time spent in the centre square very closely approached significance (p=0.056).

### Discussion (Study 1)

These results show a clear and robust anxiolytic effect of the benzodiazepine, chlordiazepoxide, at 2.5 mg/kg, as previously reported (File and Pellow, 1987). This effect was observed in several aspects of behaviour on the elevated plus maze. CDP significantly increased the number of entries into, and time spent on, the open arms of the maze. This area is thought to represent an aversive environment for the rats as it is open and provides no shelter from potential predators and sources of threat. Control animals spent approximately 4 times longer on the closed arms compared with the open arms (FIG. 1.3; 2.3) which is predicted as the walls around the closed arms provide some protection from potential predators. CDP had no significant effect on the number of rears or time spent in the centre square, although a trend towards a reduction in time spent in the centre square was observed. The centre square is a transition place from closed to open arms and animals will spend time in risk assessment in this part of the maze, a reduction in time spent in this area may be viewed as a reduction in risk assessment behaviour which would be expected from an agent reducing anxiety levels. It would be expected that this behaviour would now be seen as an increase in time spent on the open arm of the maze, which was in fact produced by CDP. CDP also reduced time spent on the closed arms, although this effect failed to achieve statistical significance, FIG. 2.4.

Isomer A showed some anxiolytic potential in this study, in some measures of behaviour on the elevated plus maze. Isomer A at 1 mg/kg significantly increased the time spent on the open arms with an increase in the number of entries into the open arms, although this effect failed to achieve statistical significance. The magnitude of the increase in time spent on the open arms following 1 mg/kg of Isomer A was comparable to that produced by CDP at 2.5 mg/kg, FIGS. 1.3 and 2.3. Isomer A at 1 mg/kg also reduced time spent on the closed arms, although again this effect was not statistically significant. The effect of 1 mg/kg Isomer A to increase time spent on the open arms suggests that this compound may have some efficacy to reduce anxiety in female hooded-Lister rats, in a manner comparable, to that of CDP.

In Study 1, only the lowest dose tested had the effect of making the animals calmer and easier to handle which could be associated with reduced anxiety levels. In order to clarify why this was so, a follow up study was carried out (Study 2).

### Study 2

The results obtained from study 2 are shown in FIGS. 3 to 8.

FIG. 3 illustrates the effect of chlordiazepoxide treatment (CDP 2.5 mg/kg, i.p.) and Isomer A (0.1-2.5 mg/kg, p.o.) on total open arm entries in a 10 min trial on the elevated plus-maze. Data are expressed as mean±s.e.m. (n=7-10 per group) and were analysed by ANOVA and post-hoc Dunnett's t-test. The data demonstrate that there was no overall significant effect of CDP and Isomer A on total entries into the open arms of the elevated plus maze [F(5,48)=1.79, NS].

FIG. 4 illustrates the effect of chlordiazepoxide treatment (CDP 2.5 mg/kg, i.p.) and Isomer A (0.1-2.5 mg/kg, p.o.) on total closed arm entries in a 10 min trial on the elevated plus maze. Data are expressed as mean±s.e.m. (n=7-10 per group) and were analysed by ANOVA and post-hoc Dunnett's t-test. The data demonstrate that there was no overall significant effect of CDP and Isomer A on total entries into the closed arms of the elevated plus maze [F(5,48)=1.58, NS].

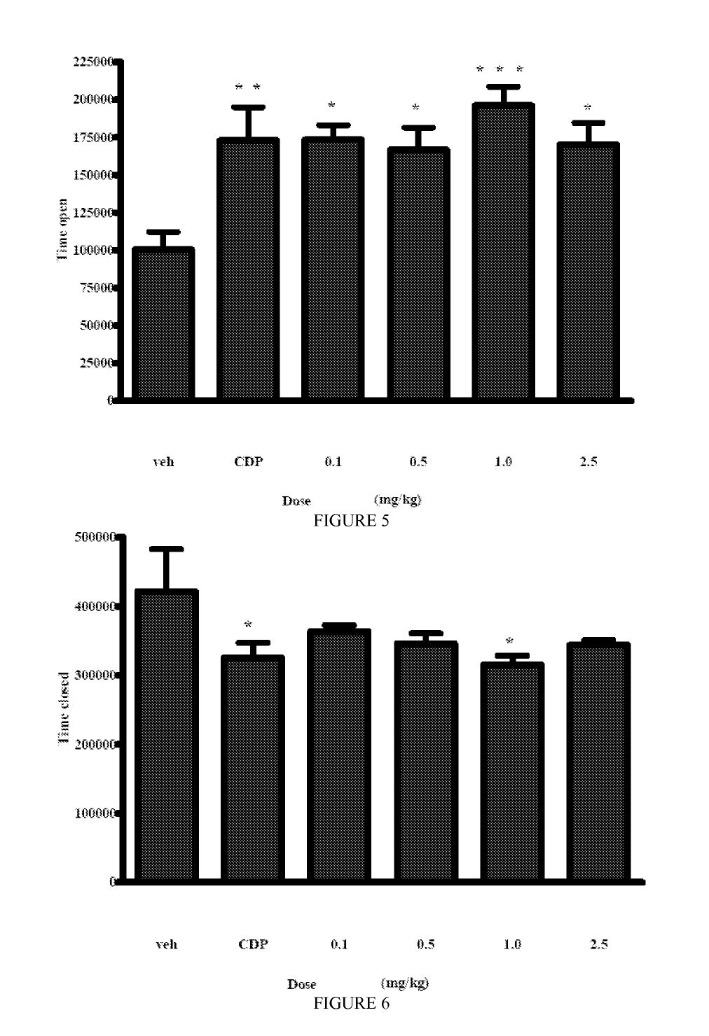

FIG. 5 illustrates the effect of chlordiazepoxide treatment (CDP 2.5 mg/kg, i.p.) and Isomer A (0.1-2.5 mg/kg, p.o.) on time spent in the open arms in a 10 min trial on the elevated plus maze. Data are expressed as mean±s.e.m. (n=7-10 per group) and were analysed by ANOVA and post-hoc Dunnett's t-test (*P<0.05-***P<0.001; significant increase in time spent on the open arms compared to vehicle). The data demonstrate that there was an overall significant effect of CDP and Isomer A on time spent on the open arms of the elevated plus maze [F(5,48)=4.19, p<0.005]. Post-hoc analysis revealed that, at all doses of Isomer A, there was a significant increase in time spent on the open arms of the elevated plus maze (p<0.05-p<0.001), FIG. 3. CDP also significantly increased time spent on the open arms (p<0.01).

FIG. 6 illustrates the effect of chlordiazepoxide treatment (CDP 2.5 mg/kg, i.p.) and Isomer A (0.1-2.5 mg/kg, p.o.) on time spent in the closed arms in a 10 min trial on the elevated plus-maze. Data are expressed as mean±s.e.m. (n=7-10 per group) and were analysed by ANOVA and post-hoc Dunnett's t-test (*P<0.05; significant reduction in time spent on the closed arms compared to vehicle). The data demonstrate that there was an overall significant effect of CDP and Isomer A on time spent on the closed arms of the elevated plus maze [F(5,48)=4.74, p<0.005]. Post-hoc analysis revealed that, at 1.0 mg/kg of Isomer A, there was a significant reduction in time spent on the closed arms of the elevated plus maze (p<0.05). CDP also significantly reduced time spent on the closed arms (p<0.05).

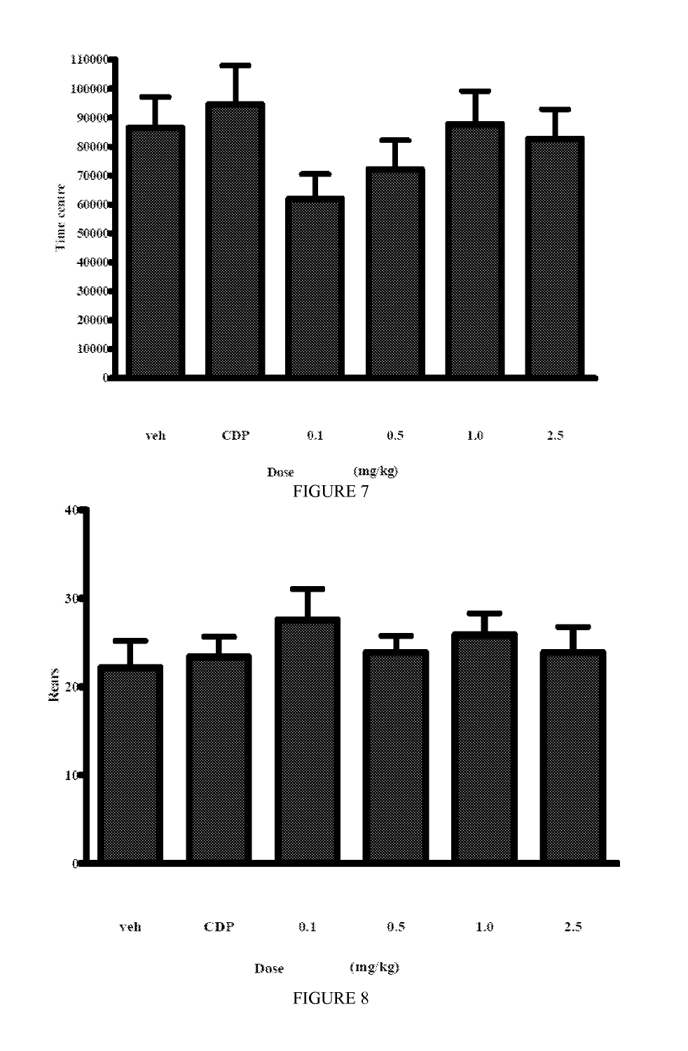

FIG. 7 illustrates the effect of chlordiazepoxide treatment (CDP 2.5 mg/kg, i.p.) and Isomer A (0.1-2.5 mg/kg, p.o.) on time spent in the centre square in a 10 min trial on the elevated plus-maze. Data are expressed as mean±s.e.m. (n=7-10 per group) and were analysed by ANOVA and post-hoc Dunnett's t-test. The data demonstrate that there was no overall significant effect of CDP and Isomer A on time spent in the centre square of the elevated plus maze [F(5,48)=1.18, NS].

FIG. 8 illustrates the effect of chlordiazepoxide treatment (CDP 2.5 mg/kg, i.p.) and Isomer A (0.1-2.5 mg/kg, p.o.) on the number of rears in a 10 min trial on the elevated plus-maze. Data are expressed as mean±s.e.m. (n=7-10 per group) and were analysed by ANOVA and post-hoc Dunnett's t-test. The data demonstrate that there was no overall significant effect of CDP and Isomer A on total rears on the elevated plus maze [F(5,48)=0.5, NS].

### Discussion (Study 2)

These results show a clear and robust anxiolytic effect of the benzodiazepine, chlordiazepoxide, at 2.5 mg/kg, as previously reported (File and Pellow, 1987; and see also Study 1 above. This effect was observed in several aspects of behaviour on the elevated plus maze. CDP increased the number of entries into, and significantly increased time spent on, the open arms of the maze with a concomitant significant reduction in time spent on the closed arms of the maze. The open area is thought to represent an aversive environment for the rats as it is exposed and provides no shelter from potential predators and sources of threat. Control animals spent approximately 4 times longer on the closed arms compared with the open arms (FIGS. 5; 6) which is predicted as the walls around the closed arms provide some protection from potential predators. CDP had no significant effect on the number of rears or time spent in the centre square.

Isomer A again showed anxiolytic potential in this study, at all doses tested, 0.1-2.5 mg/kg, in certain measures of behaviour on the elevated plus maze. Isomer A at all doses tested significantly increased the time spent on the open arms with no effect on the number of entries into the open arms. The magnitude of the increase in time spent on the open arms following Isomer A was comparable to that produced by CDP at 2.5 mg/kg, FIG. 5. Isomer A at 1 mg/kg also significantly reduced time spent on the closed arms.

The effect of Isomer A to increase time spent on the open arms suggests that this compound may have some efficacy to reduce anxiety in female hooded-Lister rats, in a manner comparable to that of CDP. This effect appeared most robust at a dose of 1 mg/kg: indeed, Study 1 showed an anxiolytic profile at this dose in the same paradigm. Isomer A at 0.1 mg/kg, the lowest dose tested, reduced time in the centre square, although this effect failed to achieve statistical significance. The centre square is a transition place from closed to open arms and animals will spend time in risk assessment in this part of the maze, a reduction in time spent in this area may be viewed as a reduction in risk assessment behaviour which would be expected from an agent reducing anxiety levels. It would be expected that this behaviour would now be seen as an increase in time spent on the open arm of the maze, which was in fact produced by Isomer A, at all doses tested.

In summary, based on the results obtained from Studies 1 and 2 above, Isomer A is expected to be useful as an anxiolytic drug.

### Example 4

### Pharmaceutical Compositions

### (i) Tablet Formulation-I

A tablet composition containing the dihydrotetrabenazine of the invention is prepared by mixing 50 mg of the dihydrotetrabenazine with 197 mg of lactose (BP) as diluent, and 3 mg magnesium stearate as a lubricant and compressing to form a tablet in known manner.

(ii) Tablet Formulation-II A tablet composition containing the dihydrotetrabenazine of the invention is prepared by mixing the compound (25 mg) with iron oxide, lactose, magnesium stearate, starch maize white and talc, and compressing to form a tablet in known manner.
(iii) Capsule Formulation

A capsule formulation is prepared by mixing 100 mg of the dihydrotetrabenazine of the invention with 100 mg lactose and filling the resulting mixture into standard opaque hard gelatin capsules.

### EQUIVALENTS

It will readily be apparent that numerous modifications and alterations may be made to the specific embodiments of the invention described above without departing from the principles underlying the invention. All such modifications and alterations are intended to be embraced by this application.

1-10. (canceled)

11. A method for the prophylaxis or treatment of anxiety comprising administering 3,11b-cis-dihydrotetrabenazine of the formula (Ib): 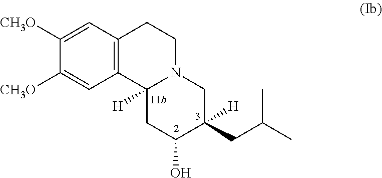
or a pharmaceutically acceptable salt thereof.

12. The method of claim 11 wherein the 3,11b-cis-dihydrotetrabenazine is in the form of an acid addition salt.

13. The method of claim 11 wherein the 3,11b-cis-dihydrotetrabenazine salt is a methane sulphonate salt.

14. The method of claim 11 wherein the anxiety is secondary to other psychiatric illness.

15. The method of claim 11 wherein the anxiety is primary anxiety.

16. The method of claim 15 wherein the anxiety is associated with a neurosis.

17. The method of claim 15 wherein the anxiety is phobic anxiety.

18. The method of claim 17 wherein the phobic anxiety arises from social phobias, general phobias and specific phobias.

19. The method of claim 11 wherein the anxiety arises from an obsessional disorder.

20. The method of claim 11, wherein the 3,11b-cis-dihydrotetrabenazine of the formula (Ib) is administered in the form of a pharmaceutical composition.
